(19) United States
(12) Patent Application Publication
(10) Pub. No.: US 2004/0019219 A1
(43) Pub. Date: Jan. 29, 2004

(54) N-alkylated thiazolium salts and process for their preparation

(76) Inventors: Ralf Wischnat, Koln (DE); Joachim Rudolph, Guilford, CT (US)

Correspondence Address: BAYER CHEMICALS CORPORATION, 100 BAYER ROAD, PITTSBURGH, PA, 15205, US

(21) Appl. No.: 10/458,092

(22) Filed: Jun. 10, 2003

(30) Foreign Application Priority Data
Jun. 10, 2002 (DE) 10225537.7

(51) Int. Cl. C07D 513/02; C07D 513/12; C07D 277/20

(52) U.S. Cl. 548/151; 548/153; 548/202

(57) ABSTRACT

The present invention relates to N-alkylated thiazolium salts, to a process for their preparation and also to their use as condensation and dehydrating agents.

### BACKGROUND OF THE INVENTION

1. Field of the Invention

The present invention relates to N-alkylated thiazolium salts, to a process for their preparation and also to their use as condensation and dehydrating agents.

2. Brief Description of the Prior Art

BEMT, 2-bromo-3-ethyl-4-methylthiazolium tetrafluoroborate, an N-alkylated thiazolium salt, has found use as a peptide coupling reagent. It is customarily prepared by alkylating 2-bromo-4-methylthiazole with triethyloxonium tetrafluoroborate, also known as Meerwein salt (see P. Li, Tetrahedron Lett. 1999, 40, 8301-8304). The 2-bromo-4-methylthiazole used as a starting product is in turn prepared by a Sandmeyer reaction starting from 2-amino-4-methylthiazole.

However, the disadvantage of this synthetic method is that the preparation of 2-bromo-4-methylthiazole is only possible in small yields and the alkylation entails the use of the expensive triethyloxonium tetrafluoroborate.

There was therefore a need to provide an efficient process which facilitates the N-alkylation of thiazoles in good yields.

Equally, there was a need for further N-alkylated thiazolium salts which can be used with high efficiency, in particular as condensation and dehydrating agents.

### SUMMARY OF THE INVENTION

A process has now been found for preparing N-alkylated thiazolium salts, which is characterized in that thiazoles of the formula (I)

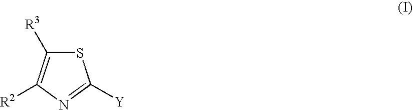

where

R² is hydrogen, C₁-C₁₂-alkyl, C₄-C₁₄-aryl or C₅-C₁₅-arylalkyl and

R³ is hydrogen, C₁-C₁₂-alkyl, C₄-C₁₄-aryl or C₅-C₁₅-arylalkyl or

R² and R³ together represent the following groups

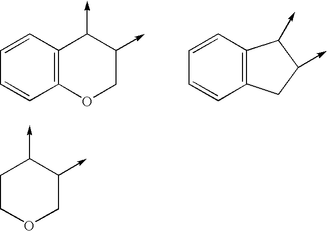

where the arrows indicate the linking points with the thiazole ring, or R² and R³ together are radicals which are selected from the group of 1,3-propanediyl, 1,4-butanediyl or 1,5-pentanediyl, and each of the groups and radicals mentioned may also be mono- or polysubstituted by halogen, nitro, formyl, C₁-C₆-alkyl, C₁-C₆-haloalkyl, C₁-C₆-alkoxy or C₁-C₆-haloalkoxy and

Y is iodine, bromine or chlorine

are reacted in the presence of a solvent with componds of the formula (II)

R¹—X (II)

where

R¹ is primary or secondary C₁-C₃-alkyl, primary or secondary C₄-C₁₂-alkyl or C₅-C₁₅-arylalkyl and

X is chlorine, bromine or iodine.

This method provides compounds of the formula (III)

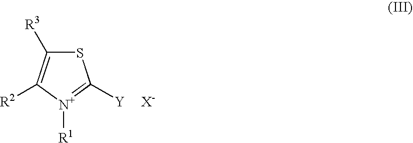

where

R¹, R² and R³ are each as defined under formula (I) and X is as defined under formula (II).

Within the scope of the invention, the above-listed general radical definitions or illustrations and parameters, or those listed within areas of preference, i.e. the particular areas and areas of preference, may be combined as desired.

### DETAILED DESCRIPTION OF THE INVENTION

For the purposes of the invention, alkyl and alkoxy are each independently a straight-chain or cyclic, and independently thereof, branched or unbranched, alkyl or alkoxy radical which may be further substituted typically by C₁-C₄-alkoxy radicals. The alkyl moiety of an arylalkyl radical is the same as the alkyl described above. For the purposes of the invention, for example, C₁-C₃-alkyl is in each case methyl, ethyl, 2-ethoxyethyl, n-propyl and isopropyl; C₁-C₄-alkyl is additionally n-butyl and tert-butyl; C₁-C₆-alkyl is additionally, for example, n-pentyl, cyclohexyl and n-hexyl; C₁-C₁₂-alkyl is further additionally n-heptyl, n-octyl, isooctyl, norbornyl, n-decyl and n-dodecyl. In particular, primary or secondary C₄-C₁₂-alkyl is n-butyl, sec-butyl, n-pentyl, neopentyl, n-hexyl, cyclohexyl, n-heptyl, n-octyl, n-decyl and n-dodecyl.

For example, C₁-C₄-alkoxy is in each case methoxy, ethoxy, 2-ethoxyethoxy, n-propoxy, isopropoxy, n-butoxy and tert-butoxy, and C₁-C₆-alkoxy is additionally, for example, n-pentoxy, cyclohexoxy and n-hexoxy.

For the purposes of the invention, haloalkyl or haloalkoxy are each independently a straight-chain or cyclic, and independently thereof branched or unbranched, alkyl or alkoxy radical as defined above, the radicals each being substituted singly, multiply or fully by halogen atoms, preferably chlorine and/or fluorine atoms.

C₁-C₆-Haloalkyl is, for example, trifluoromethyl, trichloromethyl, 2,2,2-trifluoro-ethyl, pentafluoroethyl and nonafluorobutyl, C₁-C₆-haloalkoxy is, for example, trifluoromethoxy, pentafluoroethoxy or 2,2,2-trifluoroethoxy.

For the purposes of the invention, aryl is, for example and with preference, a carbocyclic aromatic radical or heteroaromatic radical which bears no, one, two or three heteroatoms per cycle, but at least one heteroatom in the entire heteroaromatic radical, which is selected from the group of nitrogen, sulphur and oxygen.

The carbocyclic aromatic radicals or heteroaromatic radicals may also be substituted by one, two or three substituents per cycle which are each independently selected from the group of nitro, C₁-C₄-alkylsulphonyl, C₁-C₄-alkoxycarbonyl, C₁-C₄-haloalkoxycarbonyl, C₁-C₄-alkylcarbonyloxy or C₁-C₄-haloalkylcarbonyloxy, C₁-C₆-alkyl, cyano, COO—(C₁-C₁₆-alkyl), COO—(C₄-C₁₀-aryl), CO—(C₁-C₆-alkyl), CO—(C₄-C₁₀-aryl), O—(C₁-C₆-alkyl), O—(C₄-C₁₀-aryl), N(C₁-C₆-alkyl)₂, fluorine, chlorine, bromine and C₁-C₆-haloalkyl. The same applies to the aryl moiety of an arylalkyl radical.

In formula (III)

R¹ is preferably methyl, ethyl, n-propyl, isopropyl, n-butyl or benzyl which is optionally mono- or polysubstituted by fluorine and/or chlorine, methyl, ethyl, n- or isopropyl, trifluoromethyl, methoxy or ethoxy, particularly preferably methyl, ethyl, n-propyl or benzyl and very particularly preferably methyl, ethyl and n-propyl,

R² is preferably hydrogen, methyl, ethyl, n-propyl, isopropyl, n-butyl or sec-butyl, or benzyl or phenyl, both of which are optionally mono- or polysubstituted by fluorine and/or chlorine, methyl, ethyl, n- or isopropyl, methoxy, ethoxy or n- or isopropoxy, particularly preferably methyl, ethyl, n-propyl, n-butyl or phenyl which is optionally mono- or polysubstituted by fluorine and/or chlorine, methyl or ethyl, and very particularly preferably methyl or ethyl,

R³ is preferably hydrogen, methyl, ethyl or isopropyl, particularly preferably hydrogen or methyl and even more particularly preferably hydrogen,

R² and R³ together preferably represent the groups

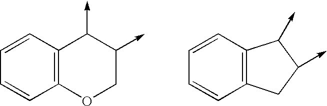

where the arrows indicate the linking points with the thiazole ring or are together 1,3-propanediyl or 1,4-butanediyl,

Y is preferably iodine or bromine, particularly preferably bromine,

X⁻ is preferably bromide and iodide, particularly preferably bromide.

Compounds of the formula (I) preferred for the process according to the invention are: 2-chloro-4-methylthiazole, 2-bromo-4-methylthiazole, 2-iodo-4-methylthiazole, 2-chlorothiazole, 2-bromothiazole, 2-iodothiazole, 2-chloro-4,5-dimethylthiazole, 2-bromo-4,5-dimethylthiazole, 2-iodo-4,5-dimethylthiazole, 2-chloro-4-ethyl-5-methylthiazole, 2-bromo-4-ethyl-5-methylthiazole and 2-iodo-4-ethyl-5-methylthiazole.

Preferred compounds of the formula (II) are:

methyl chloride, methyl bromide, methyl iodide, ethyl chloride, ethyl bromide, ethyl iodide, benzyl chloride, benzyl bromide and benzyl iodide.

The process according to the invention is carried out in the presence of solvent. Examples of useful solvents include optionally halogenated aliphatic and aromatic hydrocarbons, for example petroleum ether, hexane, heptane, cyclohexane, methylcyclohexane, benzene, toluene, xylene or decalin, chlorobenzene, dichloro-benzene, methylene chloride, chloroform, tetrachloromethane, dichloro- or trichloroethane or tetrachloroethylene, amidic solvents, for example dimethylacetamide or dimethylformamide, sulphoxides and sulphones, for example dimethyl sulphoxide or tetramethylene sulphone, or mixtures of the solvents mentioned.

Preference is given to dimethylformamide and dimethylacetamide.

In a preferred embodiment of the process according to the invention, a mole ratio of, for example, from 1.0 to 10.0 mol, preferably from 1.1 to 10.0 mol, particularly preferably from 2.1 to 10.0 mol and very particularly preferably from 2.5 to 5 mol, of compound of the formula (II) may be used per mole of the compound of the formula (I).

The concentration of the compound of the formula (II) in the solvent may be, for example and with preference, from 0.2 to 6.0 mol/l, particularly preferably from 2.5 to 6.0 mol/l.

The compound of the formula (II) may be added immediately at the beginning of the reaction or continuously, optionally in portions.

The reaction temperature may be, for example, 20 to 100° C., although preference is given to 50 to 75° C. Particular preference is given to gradually heating to 50 to 75° C. within 1 to 6 hours, preferably 1.5 to 2.5 hours.

The reaction vessel used may be a customary glass apparatus. However, it may be advantageous when, as a consequence of the boiling temperature of the reagents or of the solvent, pressures result and therefore working in pressure-resistant vessels. Examples of useful pressure vessels include inert steel vessels or vessels of steel alloys (for example Hastelloy, Va. steel or alloy).

The reaction pressure may be, for example, 1 to 150 bar, although preference is given to 3 to 10 bar, particular preference to 3 to 6 bar.

The reaction time depends on the reactivity of the compound of the formula (II) used and may be, for example, 1 to 100 hours, preferably 1 to 48 hours. Very particular preference is given to reaction times of 11 to 18 hours.

Especially in the case of compounds of the formula (II) which have a boiling point of 50° C. or below at atmospheric pressure, it is advantageous to carry out the reaction within the abovementioned preferred ranges of pressure and to compensate for the pressure drop with increasing reaction progress by injecting an inert gas, for example oxygen, nitrogen, air or argon. Good mixing may likewise have an advantageous effect on the reaction. Preference is given to stirring or agitating the mixture. Particular preference is given to mixing carried out, for example, by the use of paddle stirrers, intermix stirrers or anchor stirrers.

After cooling to room temperature, the compounds of the formula (III) may optionally be isolated by crystallization, filtration or extraction. Preference is given to isolation by filtration. Residues and impurities may be removed by washing the crystalline product, for example, with an inert solvent, for example a hydrocarbon or halohydrocarbon. The products are advantageously stored with the exclusion of moisture.

A further aspect of the invention relates to the reaction of compounds of the formula (III) with halides of the formula (IV)

M(Y²)ₙ (IV)

where

M is a metal ion having valency n or a quaternary ammonium ion, for example, tetrabutylammonium, and

Y² is chlorine, bromine or iodine to obtain compounds of the formula (IIIb)

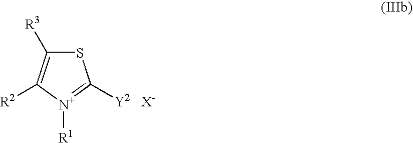

where

R¹, R², R³ and X⁻ are each as defined under formula (III) and

Y² is as defined under formula (IV).

The process is suitable in particular for converting compounds of the formula (III) where Y is chlorine or bromine to compounds of the formula (IIIb) where Y² is iodine, or for converting compounds of the formula (III) where Y is chlorine to compounds of the formula (IIIb) where Y² is bromine or iodine.

Preferred compounds of the formula (IV) are alkali metal, alkaline earth metal and transition metal chlorides, bromides and iodides, particular preference is given to the chlorides, bromides and iodides of lithium, sodium, potassium, magnesium, calcium, iron(III), zinc(II) and copper(II), and even greater preference is given to the bromides and iodides mentioned.

A further aspect of the invention relates to the preparation by anion exchange of compounds of the formula (IIIc)

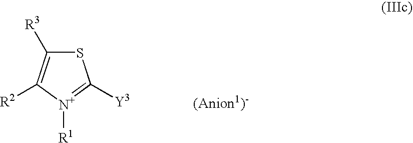

where

R¹, R² and R³ are each as defined under the formula (III) and

Y³ is chlorine, bromine or iodine and

(Anion¹)⁻ is an anion of an inorganic acid or of a sulphonic acid, with the exception of halides.

(Anion¹)⁻ is preferably tetrafluoroborate, hexafluorophosphate, half an equivalent of sulphate and sulphonate of the type R⁴SO₃⁻, where R⁴ is C₁-C₆-alkyl, C₁-C₆-haloalkyl or C₄-C₁₀-aryl as defined above.

(Anion¹)⁻ is particularly preferably tetrafluoroborate and hexafluorophosphate, and even greater preference is given to tetrafluoroborate.

The anion exchange may advantageously be effected in such a way that an anion exchanger is laden with acids of the type H(anion¹) where (anion¹) is as defined above, including the areas of preference specified, and is reacted with the compounds of the formulae (III) or (IIIb).

The invention also encompasses the hitherto unknown thiazolium salts of the formula (IIId)

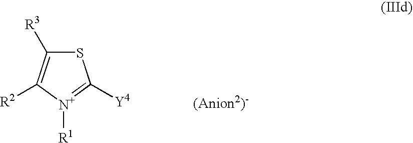

where

R² and R³ each have the definition and areas of preference specified under the formula (I) and

Y⁴ is chlorine, bromine or iodine and,

in the case that

Y⁴ is chlorine or bromine,

R¹ is primary or secondary C₄-C₁₂-alkyl and,

in the case that

Y⁴ is iodine,

R¹ is primary or secondary C₁-C₃-alkyl, primary or secondary C₄-C₁₂-alkyl or C₅-C₁₅-arylalkyl,

and the same areas of preference also apply for R¹ as specified under the formula (II)

and

(anion²)⁻ is an anion of an inorganic acid or of a sulphonic acid. (anion²)⁻ is preferably chloride, bromide, iodide, tetrafluoroborate, hexafluorophosphate, half an equivalent of sulphate or sulphonate of the type R⁴SO₃⁻, where R⁴ is C₁-C₆-alkyl, C₁-C₆-haloalkyl or C₄-C₁₀-aryl as defined above, particularly preferably chloride, bromide, iodide, tetrafluoroborate and hexafluorophosphate.

Compounds of the formula (IIId) include:

2-chloro-3,4-dimethylthiazolium chloride, 2-bromo-3,4-dimethylthiazolium bromide, 2-iodo-3,4-dimethylthiazolium iodide, 2-chloro-3-methylthiazolium chloride, 2-bromo-3-methylthiazolium bromide, 2-iodo-3-methylthiazolium iodide, 2-chloro-3,4,5-trimethylthiazolium chloride, 2-bromo-3,4,5-trimethylthiazolium bromide, 2-iodo-3,4,5-trimethylthiazolium iodide, 2-chloro-4-ethyl-3,5-dimethylthiazolium chloride, 2-bromo-4-ethyl-3,5-dimethylthiazolium bromide and 2-iodo-4-ethyl-3,5-dimethylthiazolium iodide.

The compounds of the formula (I) used as reactants for the preparation of compounds of the formula (III) can be particularly advantageously synthesized by cyclizing alpha-thiocyanato ketones. The invention therefore also encompasses a process, which is characterized in that the compounds of the formula (I)

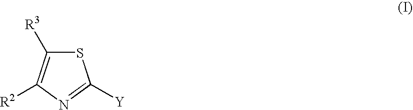

are prepared by reacting compounds of the formula (V)

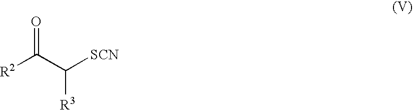

where

R² and R³ each have the same definition and areas of preference as specified under formula (I) above

with acids H(anion³) and are converted in the presence of solvent to thiazolium salts of the formula (VI)

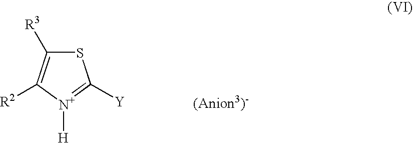

where

(Anion³)⁻ is a halide

and the compounds of the formula (VI) are converted in a further step to compounds of the formula (I) by reacting with a base.

Examples of suitable acids H(anion³) include: hydrogen chloride, hydrogen iodide and hydrogen bromide.

The compounds of the formula (IV) used as starting products are known and/or may be prepared by known processes or in a similar manner (see, for example, Schantl et al. 1998, Synth. Commun. 28, 1451-1462, Indian J. Chem., Sect. B (1991), 30, 1152-1155).

The invention also encompasses the hitherto unknown compounds of the formula (VIb)

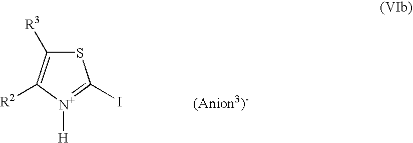

where

R² and R³ each have the same definition and areas of preference as specified under the formula (I) and

(anion³)⁻ has the same definition and areas of preference as specified above.

Individual compounds of the formula (VIb) include:

2-iodo-3,4-dimethylthiazolium iodide, 2-iodo-3-methylthiazolium iodide, 2-iodo-3,5-dimethylthiazolium iodide and 2-iodo-4-ethyl-3-methylthiazolium iodide.

Examples of useful solvents for the cyclization include: aliphatic or aromatic hydrocarbons, for example petroleum ether, hexane, heptane, cyclohexane, methylcyclohexane, benzene, toluene, xylene or decalin; halogenated hydrocarbons, for example chlorobenzene, dichlorobenzene, methylene chloride, chloroform, tetrachloromethane, dichloro-, trichloroethane, or tetrachloroethylene; ethers such as diethyl ether, diisopropyl ether, methyl t-butyl ether, methyl t-amyl-ether, dioxane, tetrahydrofuran, 1,2-dimethoxyethane, 1,2-diethoxyethane, diethylene glycol dimethyl ether or anisole. Preference is given to using methylene chloride, chloroform, 1,2-dichloroethane, diethyl ether or tert-butyl methyl ether, and particular preference is given to methylene chloride.

The amount of solvent per mole of compound of the formula (V) may be, for example, 0.5 to 51, preferably 1 to 31.

The amount of acid in the cyclization may be 2.0 to 10.0 mol, preferably 2.1 to 7 mol, per mole of a compound of the formula (V). The amounts of halide apply in a similar manner, although independently of the amount of acid.

The cyclization is advantageously carried out with the exclusion of moisture. This may be ensured, for example, by using commercially available dry diluents or drying them by the generally customary drying methods, and also using dry acids. For example, hydrogen bromide or hydrogen chloride may be passed through a refrigerated gas trap and/or a drying tower with a suitable drying agent or a gas washing apparatus, for example a wash bottle with concentrated sulphuric acid. The cyclization is advantageously carried out in such a way that the compound of the formula (IV) is preferably initially charged in the diluent and the hydrogen halide is then introduced, or the halide and then the acid are added under temperature control and good distribution. The exothermic reaction is generally carried out at a temperature of −30 to +40° C., preferably −15 to +30° C.

The resulting compounds of the formula (VI) may then be conveniently obtained by a solid-liquid separating process, for example filtering or centrifugation.

For releasing the compounds of the formula (I), organic and inorganic bases are suitable. These preferably include alkali metal carbonates or hydrogencarbonates, for example sodium, potassium or ammonium carbonate, sodium or potassium hydrogencarbonate, and also tertiary amines such as trimethylamine, triethylamine, tributylamine, N,N-dimethylaniline, N,N-dimethylbenzylamine, pyridine, N-methylpiperidine, N-methylmorpholine, N,N-dimethylaminopyridine, diazabicyclooctane (DABCO), diazabicyclononene (DBN) or diazabicycloundecene. Preference is given to sodium hydrogencarbonate or potassium hydrogencarbonate.

The compounds of the formula (I) may advantageously be released from the compounds of the formula (VI) without a preceding drying step in the same diluent as the cyclization. To this end, it is advantageous when removing the compound of the formula (VI) to wash with a little diluent, in order to substantially remove any acid present. For example, the compound of the formula (VI) is suspended in the diluent and the base is then added. Preference is given to using an aqueous solution or an inorganic base, for example sodium hydrogencarbonate solution. The concentration is uncritical. Preference is given to taking relatively highly concentrated to saturated solutions. For example, 1.0 to 1.5 mol, preferably 1.0 to 1.2 mol, of base are used per mole of the compound (V). The neutralization is generally carried out at a temperature of −20 to +30° C., preferably of −5 to +10° C.

The compounds of the formula (I) are isolated by the customary methods of organic chemistry. Preference is given to carrying out a phase separation and distilling the organic phase. Before the distillation, drying may be carried out using a drying agent, for example magnesium sulphate or sodium sulphate, calcium chloride, silica gel or molecular sieve.

The invention also encompasses the hitherto unknown thiazoles of the formula (Ib)

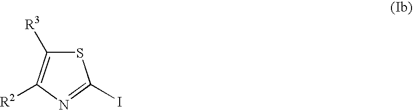

where

R² and R³ each have the same definition and areas of preference as specified under the formula (I).

The compounds of the formula (IIId), and also the compounds of the formulae (III), (IIIb) and (IIIc) which can likewise be prepared according to the invention are suitable in particular for use in condensation and dehydration reactions and also for halogenations.

For the purposes of the invention, condensation reactions refer to those reactions in which a new chemical bond is formed between two functionalities with the formal elimination of water. Preferred examples of condensation reactions are processes for preparing amidic bonds from carboxylic acids and amines, including the preparation of peptides which may optionally be cyclic, from amino acids, and also processes for preparing lactams from aminocarboxylic acids, processes for preparing esters from carboxylic acids and alcohols, including the preparation of lactones from hydroxycarboxylic acids, processes for preparing anhydrides from two identical or different carboxylic acids, including the preparation of cyclic anhydrides from dicarboxylic acids, processes for preparing isoxazoles from oximes of hydroxyketones or benzoxazinones from oximes of acylphenols, and processes for preparing oxazolines from N-(beta-hydroxyalkyl)carboxamides or thiazolines from N-(beta-hydroxyalkyl)thiocarboxamides.

For the purposes of the invention, dehydration reactions are those reactions in which the degree of bonding of an already existing chemical bond is increased with formal elimination of water. Examples include the process for preparing nitrile oxides from primary nitroalkyl compounds, processes for preparing alkenes from alcohols, such as, in particular, the preparation of α-keto olefins from α-hydroxy ketones or β-hydroxy ketones, processes for preparing nitrites from aldoximes or carboxamides, processes for preparing carbodiimides from ureas, and processes for preparing isonitriles from formyl amides.

For the purposes of the invention, halogenations are those reactions in which a hydroxyl group is converted to a halogen function. In particular, halogenations include processes for preparing alkyl fluorides, chlorides, bromides and iodides from alcohols in the presence of the corresponding halide.

Further details are evident from the examples which follow, without the invention being restricted thereto.

### PREPARATIVE EXAMPLES

### Example 1

Synthesis of 2-bromo-3,4-dialkylthiazolium bromide

100 g (0.56 mol) of 2-bromo-4-methylthiazole are dissolved 106 ml of anhydrous dimethylformamide and the mixture is introduced at room temperature into an HC autoclave. 213.28 g (2.25 mol) of bromomethane are then injected so that the pressure rises to 1.9 bar. Subsequently, the reaction mixture is heated to 70° C. at a heating rate of 8° C./minute with good mixing and the temperature is maintained at constant pressure until the conversion is substantially quantitative. The conversion is followed by mass spectrometry. For workup, the mixture is cooled to room temperature, the autoclave is decompressed and the product is filtered off. Solvent residues and impurities are removed by washing with 300 ml of hexane and the product is dried under high vacuum.

Yield: 107.5 g (70% of theory)

m.p.: 221° C.

### Example 2

Synthesis of 2-iodo-3,4-dimethylthiazolium iodide

0.55 g (2.0 mmol) of 2-bromo-3,4-dimethylthiazolium bromide (see also Example 1) are dissolved in 10 ml of dimethylformamide and admixed at room temperature with 0.33 g (2.0 mmol) of potassium iodide. For complete conversion, the mixture is heated to 50° C. for 5 h. The conversion is followed by means of EI- or FD-MS spectroscopy. After complete conversion, 2-iodo-3,4-dimethylthiazolium iodide may be used for the reactions described.

Yield: quantitative (100% of theory)

Analysis: MS

### Example 3

Synthesis of N-acetylaniline

0.3 g (0.01 mol) of acetic acid and 1.36 g (0.01 mol) of 2-bromo-3,4-dimethyl-thiazolium bromide are dissolved at −5° C. in 20 ml of dichloromethane and admixed with 1.29 g (0.01 mol) of N,N-diisopropylethylamine. Stirring is continued for 30 min and the mixture is again admixed at 0° C. with 1.29 g (0.01 mol) of N,N-diisopropylethylamine and 0.47 g (0.01 mol) of aniline. The progress of the reaction is followed by means of thin layer chromatography. For workup, the mixture is diluted with 20 ml of dichloromethane and the organic phase is washed in succession with saturated ammonium chloride solution, 1N hydrochloric acid and saturated sodium chloride solution. The organic phase is dried over magnesium sulphate and the solvent is distilled off. The residue is chromatographed on silica gel.

Yield: 0.39 g (58% of theory), white crystals

m.p.: 113-114° C.

### Example 4

Synthesis of ethyl 2-acetylamino-5-bromothiazole-4-carboxylate

0.3 g (0.01 mol) of acetic acid is dissolved at room temperature in 10 ml of dichloromethane and admixed with 1.29 g (0.01 mol) of diisopropylethylamine. 1.36 g (0.01 mol) of 2-bromo-3,4-dimethylthiazolium bromide and 1.29 g (0.01 mol) of diisopropylethylamine are added to this solution. Stirring is continued for 10 min and the mixture is admixed with 1.26 g (0.01 mol) of ethyl 2-amino-5-bromothiazole-4-carboxylate. For workup, the mixture is diluted with 20 ml of dichloromethane and the organic phase is washed with 1 N HCl, and saturated sodium chloride solution. The organic phase is dried over MgSO₄ and the solvent is distilled off on a rotary evaporator. The residue is chromatographed on silica gel using hexane/ethyl acetate in a gradient from 2:1 to 1:1.

Yield: 270 mg (19%)

m.p.: 201-203° C.

### Example 5

Synthesis of benzoic anhydride

0.61 g (5 mmol) of benzoic acid in 10 ml of dichloromethane is admixed at 0° C. with 1.29 g (10 mmol) of diisopropylethylamine, followed by 1.37 g (5 mmol) of 2-bromo-3,4-dimethylthiazolium bromide. The progress of the reaction is followed by means of thin layer chromatography. For workup, the mixture is diluted with 20 ml of dichloromethane and the organic phase is washed in succession with 1 N HCl and saturated sodium chloride solution. The solvent is dried over MgSO₄ and distilled off after filtration. For workup, the mixture is filtered through silica gel using dichloromethane as the eluent. To isolate the pure product, the solvent is distilled off.

Yield: 0.36 g (64% of theory)

GC (MS/Cl): calc. m/z=226.23, found: m/z=226

### Example 6

Synthesis of Z-Phg-Ala-OMe

1.5 g (5.26 mmol) of Z-(S)-phenylglycine are dissolved at −5° C. in 10 ml of abs. dichloromethane and admixed with 1.36 g (10.52 mmol) of ethyl diisopropylamine, followed by 1.44 g (5.26 mmol) of 2-bromo-3,4-dimethylthiazolium bromide. Stirring is continued at 0° C. for 30 min and the mixture is admixed with a mixture of 0.73 g (5.26 mmol) of L-alanine methyl ester hydrochloride and 0.68 g (5.26 mmol) of ethyl diisopropylamine in 10 ml of dichloromethane. Stirring is continued at 0° C. for 2 h and, for workup, the mixture is admixed with 20 ml of dichloromethane and the organic phase is washed in succession with saturated ammonium chloride solution and saturated sodium chloride solution. The organic phase is dried over MgSO₄ and the solvent is distilled off on a rotary evaporator. The residue is filtered through silica gel using 3:7 ethyl acetate/hexane.

Yield: 0.6 g (31%)

1H NMR (400 MHz, CDCl₃): 1.42 (d, 3H, CH₃—Ala, J=7.1 Hz), 3.72 (s, 3H, OCH₃), 4.55 (m, 1H, CHCH₃), 5.01-5.3 (m, br, 3H), 6.01 (s, br, NH), 6.3 (m, br, NH), 7.1-7.6 (m, 10H, aromatic).

Although the invention has been described in detail in the foregoing for the purpose of illustration, it is to be understood that such detail is solely for that purpose and that variations can be made therein by those skilled in the art without departing from the spirit and scope of the invention except as it may be limited by the claims.

What is claimed is:

1. Process for preparing N-alkylated thiazolium salts, comprising reacting thiazoles of the formula (I) 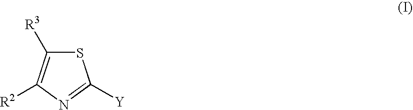
where
R² is hydrogen, C₁-C₁₂-alkyl, C₄-C₁₄-aryl or C₅-C₁₅-arylalkyl and
R³ is hydrogen, C₁-C₁₂-alkyl, C₄-C₁₄-aryl or C₅-C₁₅-arylalkyl or
R² and R³ together represent the following groups 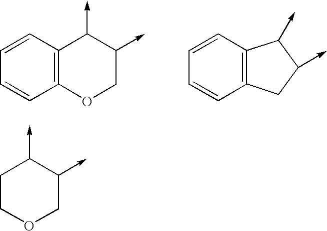
where the arrows indicate the linking points with the thiazole ring, or R² and R³ together are radicals which are selected from the group of 1,3-propanediyl, 1,4-butanediyl or 1,5-pentanediyl, and the groups and radicals mentioned are optionally mono- or polysubstituted by halogen, nitro, formyl, C₁-C₆-alkyl, C₁-C₆-haloalkyl, C₁-C₆-alkoxy or C₁-C₆-haloalkoxy and
Y is iodine, bromine or chlorine
in the presence of a solvent with componds of the formula (II)
R¹—X (II)
where
R¹ is primary or secondary C₁-C₃-alkyl, primary or secondary C₄-C₁₂-alkyl or C₅-C₁₅-arylalkyl
X is chlorine, bromine or iodine
characterized in that the N-alkylated thiazolium salts are of the formula (III) 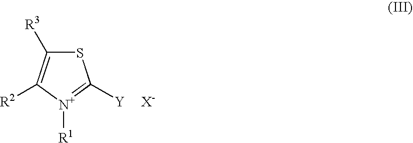
where
R¹, R² and R³ are each as defined under formula (I) and X is as defined under formula (II).

2. Process according to claim 1, characterized in that
R¹ is methyl, ethyl, n-propyl, isopropyl, n-butyl or benzyl which is optionally mono- or polysubstituted by fluorine or chlorine, methyl, ethyl, n- or isopropyl, trifluoromethyl, methoxy or ethoxy.

3. Process according to claim 1, characterized in that
R² is methyl, ethyl, n-propyl, isopropyl, n-butyl or sec-butyl, or benzyl or phenyl, both of which are optionally mono- or polysubstituted by fluorine or chlorine, methyl, ethyl, n- or isopropyl, methoxy, ethoxy or n- or isopropoxy.

4. Process according to claim 1, characterized in that
R³ is hydrogen, methyl, ethyl or isopropyl.

5. Process according to claim 1, characterized in that
R² and R³ together represent the groups 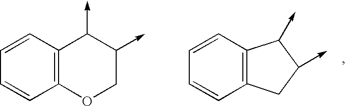
where the arrows indicate the linking points with the thiazole ring or together represent 1,3-propanediyl or 1,4-butanediyl.

6. Process according to claim 1, characterized in that the compounds of the formula (I) used are 2-chloro-4-methylthiazole, 2-bromo-4-methylthiazole, 2-iodo-4-methylthiazole, 2-chlorothiazole, 2-bromothiazole, 2-iodothiazole, 2-chloro-4,5-dimethylthiazole, 2-bromo-4,5-dimethylthiazole, 2-iodo-4,5-dimethylthiazole, 2-chloro-4-ethyl-5-methylthiazole, 2-bromo-4-ethyl-5-methylthiazole or 2-iodo-4-ethyl-5-methylthiazole.

7. Process according to claim 1, characterized in that the compounds of the formula (II) used are methyl chloride, methyl bromide, methyl iodide, ethyl chloride, ethyl bromide, ethyl iodide, benzyl chloride, benzyl bromide or benzyl iodide.

8. Process according to claim 1, characterized in that the solvent used is an optionally halogenated aliphatic or aromatic hydrocarbon, amidic solvent, sulphoxide or sulphone, or a mixture of the solvents mentioned.

9. Process according to claim 1, characterized in that the reaction pressure is from 1 to 150 bar.

10. Process for preparing compounds of the formula (IIIb) 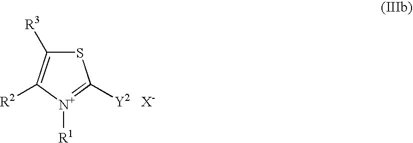
where
R² is hydrogen, C₁-C₁₂-alkyl, C₄-C₁₄-aryl or C₅-C₁₅-arylalkyl and
R³ is hydrogen, C₁-C₁₂-alkyl, C₄-C₁₄-aryl or C₅-C₁₅-arylalkyl or
R² and R³ together represent the following groups 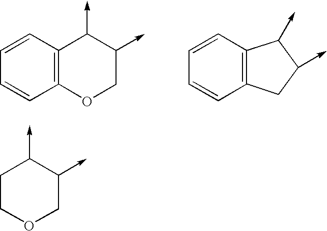
where the arrows indicate the linking points with the thiazole ring, or R² and R³ together are radicals which are selected from the group of 1,3-propanediyl, 1,4-butanediyl or 1,5-pentanediyl, and the groups and radicals mentioned are optionally mono- or polysubstituted by halogen, nitro, formyl, C₁-C₆-alkyl, C₁-C₆-haloalkyl, C₁-C₆-alkoxy or C₁-C₆-haloalkoxy and
Y² is iodine, bromine or chlorine and
X is chlorine, bromine or iodine,
comprising reacting compounds of the formula (III) 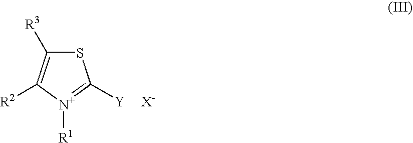
where
R¹, R² and R³ are each as defined under formula (I) and X is as defined under formula (II)
with halides of the formula (IV)
M(Y²)ₙ (IV)
where
M is a metal ion having a valency n or a tertiary ammonium ion and
Y² is as defined under formula (IIIb).

11. Process according to claim 10, characterized in that the halides of the formula (IV) used are alkali metal, alkaline earth metal and transition metal chlorides, bromides or iodides.

12. Process for preparing compounds of the formula (IIIc) 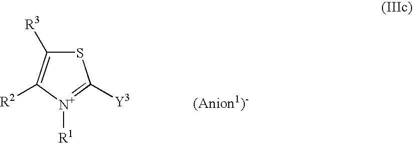
where
R² is hydrogen, C₁-C₁₂-alkyl, C₄-C₁₄-aryl or C₅-C₁₅-arylalkyl and
R³ is hydrogen, C₁-C₁₂-alkyl, C₄-C₁₄-aryl or C₅-C₁₅-arylalkyl or
R² and R³ together represent the following groups 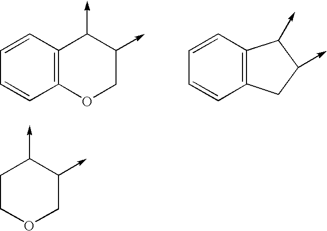
where the arrows indicate the linking points with the thiazole ring, or R² and R³ together are radicals which are selected from the group of 1,3-propanediyl, 1,4-butanediyl or 1,5-pentanediyl, and the groups and radicals mentioned are optionally mono- or polysubstituted by halogen, nitro, formyl, C₁-C₆-alkyl, C₁-C₆-haloalkyl, C₁-C₆-alkoxy or C₁-C₆-haloalkoxy and
Y³ is chlorine, bromine or iodine and
(anion¹)⁻ is an anion of an acid or of a sulphonic acid, comprising effecting an anion exchange.

13. Process according to claim 12, characterized in that the anion exchange is effected to provide an anion exchanger laden with acids of the type H(anion¹) where (anion¹) is as defined in claim 12 and is reacted with compounds of the formulae (III) or (IIIb).

14. Thiazolium salts of the formula (IIId) 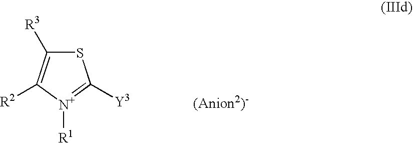
where
R² is hydrogen, C₁-C₁₂-alkyl, C₄-C₁₄-aryl or C₅-C₁₅-arylalkyl and
R³ is hydrogen, C₁-C₁₂-alkyl, C₄-C₁₄-aryl or C₅-C₁₅-arylalkyl or
R² and R³ together represent the following groups 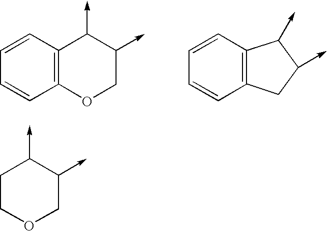
where the arrows indicate the linking points with the thiazole ring, or R² and R³ together are radicals which are selected from the group of 1,3-propanediyl, 1,4-butanediyl or 1,5-pentanediyl, and the groups and radicals mentioned are optionally mono- or polysubstituted by halogen, nitro, formyl, C₁-C₆-alkyl, C₁-C₆-haloalkyl, C₁-C₆-alkoxy or C₁-C₆-haloalkoxy and
Y³ is chlorine, bromine or iodine and,
in the case that
Y³ is chlorine or bromine,
R¹ is primary or secondary C₄-C₁₂-alkyl and,
in the case that
Y³ is iodine
R¹ is primary or secondary C₁-C₃-alkyl, primary or secondary C₄-C₁₂-alkyl or C₅-C₁₅-arylalkyl and
(anion²)⁻ is an anion of an inorganic acid or of a sulphonic acid.

15. The thiazolinium salt of claim 14 selected from the group consisting of 2-Chloro-3,4-dimethylthiazolium chloride, 2-bromo-3,4-dimethylthiazolium bromide, 2-iodo-3,4-dimethylthiazolium iodide, 2-chloro-3-methylthiazolium chloride, 2-bromo-3-methylthiazolium bromide, 2-iodo-3-methylthiazolium iodide, 2-chloro-3,4,5-trimethylthiazolium chloride, 2-bromo-3,4,5-trimethylthiazolium bromide, 2-iodo-3,4,5-trimethylthiazolium iodide, 2-chloro-4-ethyl, 3,5-dimethylthiazolium chloride, 2-bromo-4-ethyl-3,5-dimethylthiazolium bromide and 2-iodo-4-ethyl-3,5-dimethylthiazolium iodide.

16. Process according to claim 1, characterized in that the compounds of the formula (I) used as reactants are prepared by cyclizing alpha-thiocyanato ketones.

17. Process according to claim 16, characterized in that the compounds of the formula (I) [lacuna] are prepared by reacting compounds of the formula (V) 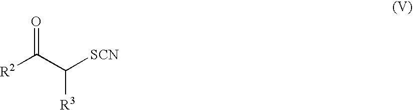
where
R² and R³ are each as defined under formula (I)
with hydrogen chloride, hydrogen bromide or hydrogen iodide, and are converted in the presence of solvent to thiazolium salts of the formula (VI) 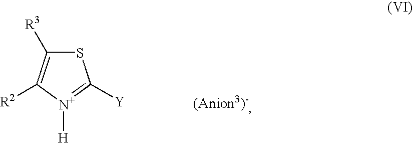
and the compounds of the formula (VI) are converted in a further step to compounds of the formula (I) by reacting with a base.

18. Compounds of the formula (VIb) 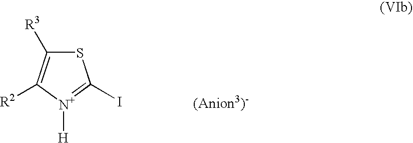
where
R² is hydrogen, C₁-C₁₂-alkyl, C₄-C₁₄-aryl or C₅-C₁₅-arylalkyl and
R³ is hydrogen, C₁-C₁₂-alkyl, C₄-C₁₄-aryl or C₅-C₁₅-arylalkyl or
R² and R³ together represent the following groups 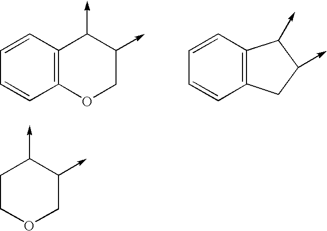
where the arrows indicate the linking points with the thiazole ring, or R² and R³ together are radicals which are selected from the group of 1,3-propanediyl, 1,4-butanediyl or 1,5-pentanediyl, and the groups and radicals mentioned are optionally mono- or polysubstituted by halogen, nitro, formyl, C₁-C₆-alkyl, C₁-C₆-haloalkyl, C₁-C₆-alkoxy or C₁-C₆-haloalkoxy and
(anion³)⁻ has the same definition and areas of preference as specified above.

19. Thiazoles of formula (VIb) selected from the group consisting of 2-iodo-3,4-dimethylthiazolium iodide, 2-iodo-3-methylthiazolium iodide, 2-iodo-3,5-dimethylthiazolium iodide and 2-iodo-4-ethyl-3-methylthiazolium iodide.

20. Thiazoles of the formula (Ib) 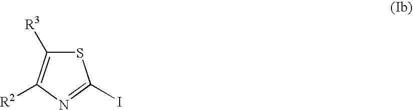
where
R² is hydrogen, C₁-C₁₂-alkyl, C₄-C₁₄-aryl or C₅-C₁₅-arylalkyl and
R³ is hydrogen, C₁-C₁₂-alkyl, C₄-C₁₄-aryl or C₅-C₁₅-arylalkyl or
R² and R³ together represent the following groups 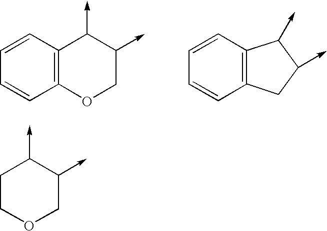
where the arrows indicate the linking points with the thiazole ring, or R² and R³ together are radicals which are selected from the group of 1,3-propanediyl, 1,4-butanediyl or 1,5-pentanediyl, and the groups and radicals mentioned are optionally mono- or polysubstituted by halogen, nitro, formyl, C₁-C₆-alkyl, C₁-C₆-haloalkyl, C₁-C₆-alkoxy or C₁-C₆-haloalkoxy.

21. A process for conducting condensation, dehydration or halogenation reactions comprising providing compounds according to claim 14 for the reactions.

22. The process according to claim 21, characterized in that condensation reactions comprise processes for preparing amidic bonds from carboxylic acids and amines.

23. The process of claim 22 wherein the preparation of amidic bonds from carboxylic acids and amines include processes for preparing peptides which are optionally cyclic, from amino acids, and processes for preparing lactams from aminocarboxylic acids, processes for preparing esters from carboxylic acids and alcohols, including the preparation of lactones from hydroxycarboxylic acids, processes for preparing anhydrides from two identical or different carboxylic acids, including the preparation of cyclic anhydrides from dicarboxylic acids, processes for preparing isoxazoles from oximes of hydroxyketones or benzoxazinones from oximes of acylphenols, and processes for preparing oxazolines from N-(beta-hydroxyalkyl)carboxamides or thiazolines from N-(beta-hydroxyalkyl)thiocarboxamides.

24. The process according to claim 21, characterized in that dehydration reactions comprise processes for preparing nitrile oxides from primary nitroalkyl compounds, processes for preparing alkenes from alcohols, processes for preparing nitrites from aldoximes or carboxamides, processes for preparing carbodiimides from ureas, or processes for preparing isonitriles from formyl amides.

25. The process according to claim 21, characterized in that halogenations comprise processes for preparing alkyl fluorides, chlorides, bromides and iodides from alcohols.
